(19) United States
(12) Patent Application Publication
(10) Pub. No.: US 2006/0167037 A1
(43) Pub. Date: Jul. 27, 2006

(54) Combinational radiotherapy and chemotherapy compositions and methods

(76) Inventors: Graham Edmund Kelly, Sydney (AU); David Brown, Sydney (AU)

Correspondence Address: SUGHRUE MION, PLLC, 2100 PENNSYLVANIA AVENUE, N.W., SUITE 800, WASHINGTON, DC, 20037, US

(21) Appl. No.: 10/547,077

(22) PCT Filed: Nov. 19, 2004
(86) PCT No.: PCT/AU04/01619

§ 371 Date: Mar. 2, 2006

(30) Foreign Application Priority Data
Nov. 19, 2003 (AU) 2003906386

(51) Int. Cl. A61K 31/4745; A61K 31/4709; A61K 31/404; A61K 31/365; A61K 31/353

(52) U.S. Cl. 514/296; 514/456; 514/454; 514/314; 514/414

(57) ABSTRACT

This invention relates to combination therapies involving radiotherapy and chemotherapy. In particular the invention relates to the use of isoflavones or analogues thereof in combination with radiotherapy or chemotherapy in the treatment of cancer and related diseases and conditions. The invention also relates to compositions and agents useful for same and methods for their manufacture.

### FIELD OF THE INVENTION

This invention relates to combination therapies involving radiotherapy and chemotherapy. In particular the invention relates to the use of isoflavones or analogues thereof in combination with radiotherapy or chemotherapy in the treatment of cancer and related diseases and conditions. The invention also relates to compositions and agents useful for same and methods for their manufacture.

### BACKGROUND

Cancer, irrespective of its pathogenesis, is characterised by uncontrolled and unregulated growth and survival of cells. This process encompasses a spectrum of change. The earliest end of this spectrum is known as pre-malignancy. At this stage, cells display a range of changes in morphology (atypical appearance, mild undifferentiation, enlargement), in mitotic activity (hyperplasia), and in survival (reduced apoptotic index). At the other end of this spectrum, cancer cells display a fully undifferentiated appearance, undergo rapid and repeated mitosis, undergo migration leading to invasion of surrounding and distant tissues, and a very low rate of apoptosis. The cancer state in humans and animals involves cells at any stage between these two ends of the spectrum.

While the exact mechanisms involved in the pathogenesis of cancer are not fully defined, an underlying error that is common to almost all forms of cancer occurs in the cellular mechanisms responsible for controlling the balance between survival and death.

Death is the normal default situation for any cell, cancerous or non-cancerous. Death is initiated by two main mechanisms—(a) the death receptors (eg. Fas, TRAIL etc), which are proteins expressed on the surface of every cell and which respond to immune cells or to soluble factors in the blood, and (b) mitochondrial disruption, which generally is triggered by internal signals generated within the cell. Death occurs through either mechanism by activation of proteolytic enzymes known as caspases, which digest cellular proteins. Death via the death receptors is known as extrinsic apoptosis; death via the mitochondrial mechanism is known as intrinsic apoptosis. In order to survive, cells need to block the ever-present death receptor-initiating factors by the production of anti-apoptotic proteins such as C-FLIP, XIAP, Smac-DIABLO, and Bax. The presence of these anti-apoptotic proteins helps to prevent the passage of the pro-death signalling cascade either to or from the death receptors or the mitochondria. Activation of the receptor, sphingosine kinase, is thought to play a key role in the production within the cell of these anti-apoptotic factors. For death to occur, the cell needs to either down-regulate the production of anti-apoptotic proteins, or to increase their rate of degradation, or both.

Survival also is promoted actively in cells via the growth receptors. This embraces such receptors as the epithelial growth factor receptor, the platelet-derived growth factor receptor, the insulin-like growth factor receptor, tumour necrosis facto receptor, and the fibroblast growth factor receptor. Activation of these growth receptors by their respective growth factors of autocrine, paracrine and endocrine origins provides an important ongoing stimulus to the cell to survive through a variety of pro-survival pathways.

Cancer is associated with dysfunction in any of these pro-survival/pro-death balancing mechanisms. For example, many cancer cells over-express one or more growth receptors, resulting in over-activation of pro-survival mechanisms. Many cancers of epithelial origin, eg. lung cancer and breast cancer, express above-normal levels of the epithelial growth factor receptor. In other instances, cancer is associated with disruption of the death receptor mechanism, by down-regulation of the expression of this receptor, and/or by over-expression of the anti-apoptotic factors.

The association of cancer with dysfunctions within the cell's control mechanisms means that the successful treatment of cancer requires either correction of these dysfunctions or activation of other functions that are able to over-ride the dysfunctional systems. That is the basis of much of current chemotherapy and radiation therapy. In essence, these various treatment modalities seek to damage a cancer cell irreversibly either metabolically or physically so that the cell induces apoptosis. That cellular damage, being so severe, triggers such pro-death outcomes as the up-regulation of expression of death receptors, and/or the up-regulation of expression of autocrine-derived ligands for the death-receptors, and/or the down-regulation of expression of anti-apoptotic factors, and/or the up-regulation of pro-death factors. The end-result from any or all of these effects is activation of the caspase-mediated proteolytic process of autolysis.

The treatment of cancer is achieved through a number of modalities. The objective of these treatments commonly is to either block or slow the rate of division of the cell (cytostasis) or to cause the cells to die (cytotoxicity). Less commonly, other objectives are to inhibit the supply of nutrients required to serve the needs of the rapidly growing cancer tissue (anti-angiogenesis). Less commonly, other objectives are to impede the functional integrity of the cell to the extent that the rate of growth is impeded (signal transduction inhibition).

Those modalities whose main effect is anti-angiogenesis include:
(a) Thalidomide

Those modalities whose main effect is cytostasis include:
(a) Topoisomerase inhibitors (eg. Topotecan (Hycamtin))

Those modalities whose main effect is cytotoxicity include:
(a) those modalities whose effect is mediated primarily through DNA damage (eg. irradiation, cisplatin, carboplatin, etoposide, bleiomycin, doxorubicin, alkylating agents)
(b) those modalities whose effect is mediated primarily through disruption of microtubules (eg. taxanes, vinca alkaloids, colcichine)
(c) those modalities whose effect is mediated primarily through an anti-metabolite action (eg. methotrexate, 5-fluorouracil, hydroxyurea, cytarabine).

Those modalities whose main effect is signal transduction modulation include:
(a) those modalities whose effect is mediated primarily through inhibition of growth receptor activity (eg. protein tyrosine kinase inhibitors, antibodies acting as agonists of growth receptors, tamoxifen, anti-androgens, anti-estrogens, GnrH agonists)
(b) those modalities whose effect is mediated primarily through up-regulation of expression or activity of death receptors (eg. interferon, Fas ligand, TRAIL ligand).

For the majority of human and animal cancers, current therapies have a number of disadvantages. First, many of them are associated with dose-limiting, adverse side-effects which reflect the non-specific nature of such therapies and are associated with damage to non-cancer tissues. Symptoms include reduced bone marrow function (anemia), gastroenteric upsets (nausea, vomiting), and hair loss. Serious burns and sores (radiation therapy), loss of libido and fertility (sex hormone antagonists), and predisposition to other forms of cancer such as leukemias (radiation therapy, some drugs) are additional disadvantages.

Second, many tumors display a native insensitivity to standard anti-cancer therapies. In such cases, standard therapies either have little or no anti-cancer effect at any dose, or the dose required to exert a significant clinical effect is grossly toxic. Examples of such tumors are renal and pancreatic carcinoma, melanoma and cholangiocarcinoma. The basis of this relative insensitivity is unknown.

Third, of those tumors that display sensitivity to standard anti-cancer therapies, many subsequently develop resistance. The basis of this induced resistance is thought to be the over-expression of proteins such as p-glycoproteins which are ‘transporter proteins’ and which eject the drug from the cell before it can exert any biological effect.

Important to the subject of this invention is addressing cellular resistance to radiotherapy or the upregulation or promotion of increased sensitivity of cancerous cells or tumors to radiation therapy.

Radiotherapy (radiation therapy) uses high energy rays, usually X-rays, to kill cancer cells. X-rays are of a similar nature to visible radiation but have an extremely short wavelength of less than 100 angstroms. X-rays were first discovered in 1895 and shortly thereafter radiation has been used in medicine for diagnosis, investigation and treatment of cancerous cells and tumors. Radiotherapy is also employed in some cases pre surgery to reduce tumor size. Radiotherapy can also be used post surgery to kill residual tissue.

Treatment is generally localised, which means that the high energy rays are directed at a particular location in or on the body. In general, cancer cells are more sensitive to radiotherapy than normal cells and more of these cell types will be killed. Normal cells which are affected usually recover to repair themselves quite quickly. Difficulties present themselves where the cancerous cells to be treated become desensitised to the radiotherapy. Other problems include the proximity of the site to be treated to vital organs, such as the heart, or the spinal cord. Here the level or dose of radiation given is critical to attack the cancerous cells without damaging surrounding normal cells, tissue or organs. Any increase in the sensitivity of the cancerous cells to the radiation, restoration of sensitivity or the protection of normal cells would be a highly sought after effect.

Accordingly, there is an urgent need to develop improved anti-cancer therapies that can address the issues of inherent or acquired radio-resistance or chemo-resistance, and that can deliver an improved anti-cancer effect in a safer, better tolerated manner.

It is a preferred object of the present invention to provide pharmaceutical compositions and methods for the treatment, amelioration or prophylaxis of cancer. The present invention also seeks to provide pharmaceutical compositions and methods for targeting cancer cells for treatment, which compositions and methods provide improved pharmacological activity in terms of targeting function, protection or differentiation of normal cells from cancerous cells, improved delivery of toxic agents and/or restoration of radiation-sensitivity or chemo-sensitivity to cancer cells with inherent or acquired resistance.

### SUMMARY OF THE INVENTION

This application now describes new treatment regimes and chemotherapeutic compositions and compounds. The invention is based on the totally unexpected activity of isoflavanoid compounds in enhancing the sensitivity of cancer cells to a wide range of anti-cancer treatments (drugs or radiation therapy) with vastly different modes of action, and in restoring sensitivity to those same agents in cells that have acquired resistance to those agent and treatment methods. Still more surprisingly, it has been found that isoflavonoid compounds exhibit protective effects on non-cancer cells and tissue.

According to an aspect of the present invention there is provided a method of increasing or restoring the sensitivity of cancer cells or a tumour to radiotherapy by contacting said cells or tumour with an isoflavonoid compound of formula (I) as set out below.

According to another aspect of the present invention there is provided a method of protecting normal cells or a tissue mass from the effects of radiotherapy and/or chemotherapy by contacting said cells or tissue mass with an isoflavonoid compound of formula (I).

According to another aspect of the present invention there is provided a method of increasing or restoring the sensitivity of cancer cells or a tumour to chemotherapy by contacting said cells or tumour with an isoflavonoid compound of formula (I). In an embodiment, the patient is subjected to both radiotherapy and chemotherapy in their treatment regime. In other embodiments the active agent is a growth receptor inhibitor or death receptor stimulator.

Compounds of the general formula (I) are the isoflavanoid compounds represented by the formula: 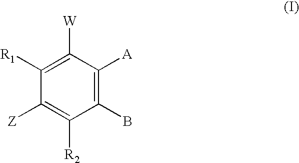
in which
R₁, R₂ and Z are independently hydrogen, hydroxy, OR₉, OC(O)R₁₀, OS(O)R₁₀, CHO, C(O)R₁₀, COOH, CO₂R₁₀, CONR₃R₄, alkyl, haloalkyl, arylalkyl, alkenyl, alkynyl, aryl, heteroaryl, alkylaryl, alkoxyaryl, thio, alkylthio, amino, alkylamino, dialkylamino, nitro or halo, or
R₂ is as previously defined, and R₁ and Z taken together with the carbon atoms to which they are attached form a five-membered ring selected from 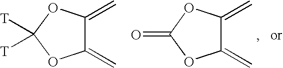
R₁ is as previously defined, and R₂ and Z taken together with the carbon atoms to which they are attached form a five-membered ring selected from 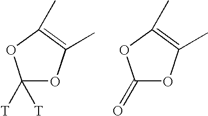
   and
W is R₁, A is hydrogen, hydroxy, NR₃R₄ or thio, and B is selected from 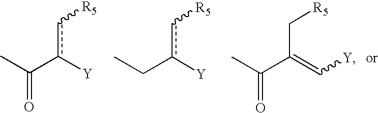
W is R₁, and A and B taken together with the carbon atoms to which they are attached form a six-membered ring selected from 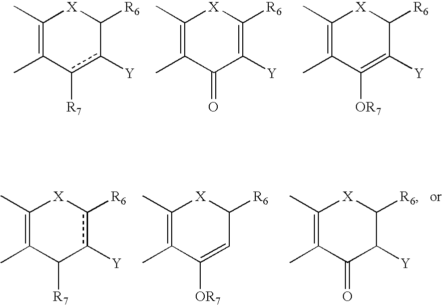
W, A and B taken together with the groups to which they are associated are selected from 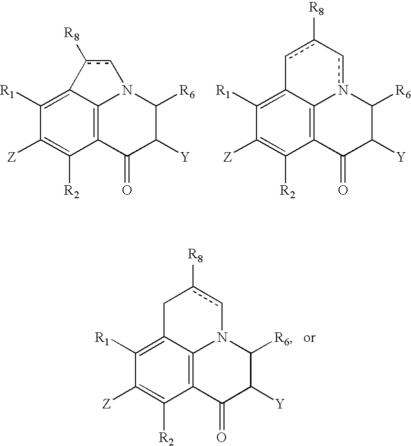
W and A taken together with the groups to which they are associated are selected from 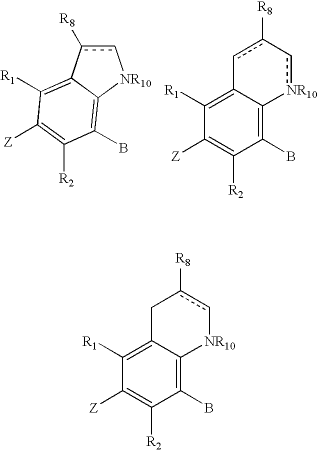
   and B is selected from 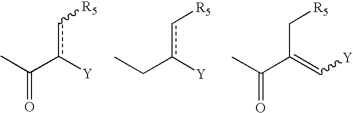
   wherein
R₃ is hydrogen, alkyl, arylalkyl, alkenyl, aryl, an amino acid, C(O)R₁₁ where R₁₁ is hydrogen, alkyl, aryl, arylalkyl or an amino acid, or CO₂R₁₂ where R₁₂ is hydrogen, alkyl, haloalkyl, aryl or arylalkyl,
R₄ is hydrogen, alkyl or aryl, or
R₃ and R₄ taken together with the nitrogen to which they are attached comprise pyrrolidinyl or piperidinyl,
R₅ is hydrogen, C(O)R₁₁ where R₁₁ is as previously defined, or CO₂R₁₂ where R₁₂ is as previously defined,
R₆ is hydrogen, hydroxy, alkyl, aryl, amino, thio, NR₃R₄, COR₁₁ where R₁₁ is as previously defined, CO₂R₁₂ where R₁₂ is as previously defined or CONR₃R₄,
R₇ is hydrogen, C(O)R₁₁ where R₁₁ is as previously defined, alkyl, haloalkyl, alkenyl, aryl, arylalkyl or Si(R₁₃)₃ where each R₁₃ is independently hydrogen, alkyl or aryl,
R₈ is hydrogen, hydroxy, alkoxy or alkyl,
R₉ is alkyl, haloalkyl, aryl, arylalkyl, C(O)R₁₁ where R₁₁ is as previously defined, or Si(R₁₃)₃ where R₁₃ is as previously defined,
R₁₀ is hydrogen, alkyl, haloalkyl, amino, aryl, arylalkyl, an amino acid, alkylamino or dialkylamino,
the drawing “ [symbol] ” represents either a single bond or a double bond,
T is independently hydrogen, alkyl or aryl,
X is O, NR₄ or S, and
Y is 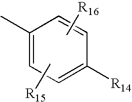
   wherein
R₁₄, R₁₅ and R₁₆ are independently hydrogen, hydroxy, OR₉, OC(O)R₁₀, OS(O)R₁₀, CHO, C(O)R₁₀, COOH, CO₂R₁₀, CONR₃R₄, alkyl, haloalkyl, arylalkyl, alkenyl, alkynyl, aryl, heteroaryl, thio, alkylthio, amino, alkylamino, dialkylamino, nitro or halo, or any two of R₁₄, R₁₅ and R₁₆ are fused together to form a cyclic alkyl, aromatic or heteroaromatic structure, and.
optionally substituted compounds and pharmaceutically acceptable salts and prodrugs thereof. In a preferred embodiment, the cancer patient is pre-treated with a compound of formula (I) prior to radiotherapy or treatment with the second chemotherapeutic agent. However it is contemplated that any sequence may be used in regard to the administration of a compound of formula (I) and either the radiotherapy or administration of the second chemotherapeutic agent or both.

In a further embodiment the compound of formula (1) is administered after resistance, either inherent or acquired, to radiotherapy is observed in a patient with cancer.

In a further embodiment the compound of formula (I) is administered after resistance, either inherent or acquired, to the chemotherapeutic agent is observed in a patient with cancer.

According to another aspect there is provided a combination therapy comprising administering to a subject undergoing radiotherapy, or about to undergo radiotherapy, a therapeutically effective amount of a compound of formula (I). In an embodiment, the administration is prior to radiotherapy and may occur after radioresistance had developed in the subject.

According to another aspect there is provided a combination therapy comprising administering to a subject a therapeutically effective amount of a compound of formula (I) and a chemotherapeutic agent. The administration may be sequential or simultaneous or after chemoresistance had developed in the subject.

In a preferred embodiment, the condition being treated is preferably cancer that is displaying malignant characteristics, but may incorporate earlier stages of cancer such as pre-malignant lesions (eg. atypia, dysplasia, intra-epitelial neoplasia) and benign cancers.

In further aspects of the invention there is provided methods for the manufacture of medicaments for the above stated methods of the invention and pharmaceutical agents useful for same.

Throughout this specification and the claims which follow, unless the text requires otherwise, the word “comprise”, and variations such as “comprises” or “comprising”, will be understood to imply the inclusion of a stated integer or step or group of integers or steps but not the exclusion of any other integer or step or group of integers or steps.

### DETAILED DESCRIPTION OF THE INVENTION

The terms “isoflavanoid”, “isoflavonoid” and “isoflavone” as used herein are to be taken broadly to include ring-fused benzopyran molecules having a pendent phenyl group from the pyran ring based on a 1,2-diphenylpropane system. Thus, the classes of compounds generally referred to as isoflavones, isoflavenes, isoflavans, isoflavanones, isoflavanols and the like are generically referred to herein as isoflavones, isoflavone derivatives or isoflavanoid compounds.

The term “alkyl” is taken to mean both straight chain and branched chain alkyl groups such as methyl, ethyl, propyl, isopropyl, butyl, isobutyl, secbutyl, tertiary butyl, and the like. The alkyl group has 1 to 10 carbon atoms, preferably from 1 to 6 carbon atoms, more preferably methyl, ethyl propyl or isopropyl. The alkyl group may optionally be substituted by one or more of fluorine, chlorine, bromine, iodine, carboxyl, C₁-C₄-alkoxycarbonyl, C₁-C₄-alkylamino-carbonyl, di-(C₁-C₄-alkyl)-amino-carbonyl, hydroxyl, C₁-C₄-alkoxy, formyloxy, C₁-C₄-alkyl-carbonyloxy, C₁-C₄-alkylthio, C₃-C₆-cycloalkyl or phenyl.

The term “aryl” is taken to include phenyl and naphthyl and may be optionally substituted by one or more C₁-C₄-alkyl, hydroxy, C₁-C₄-alkoxy, carbonyl, C₁-C₄-alkoxycarbonyl, C₁-C₄-alkylcarbonyloxy or halo.

The term “halo” is taken to include fluoro, chloro, bromo and iodo, preferably fluoro and chloro, more preferably fluoro. Reference to for example “haloalkyl” will include monohalogenated, dihalogenated and up to perhalogenated alkyl groups. Preferred haloalkyl groups are trifluoromethyl and pentafluoroethyl.

The term “pharmaceutically acceptable salt” refers to an organic or inorganic moiety that carries a charge and that can be administered in association with a pharmaceutical agent, for example, as a counter-cation or counter-anion in a salt. Pharmaceutically acceptable cations are known to those of skilled in the art, and include but are not limited to sodium, potassium, calcium, zinc and quaternary amine. Pharmaceutically acceptable anions are known to those of skill in the art, and include but are not limited to chloride, acetate, citrate, bicarbonate and carbonate.

The term “pharmaceutically acceptable derivative” or “prodrug” refers to a derivative of the active compound that upon administration to the recipient is capable of providing directly or indirectly, the parent compound or metabolite, or that exhibits activity itself. Prodrugs are included within the scope of the present invention.

As used herein, the terms “treatment”, “prophylaxis” or “prevention”, “amelioration” and the like are to be considered in their broadest context. In particular, the term “treatment” does not necessarily imply that an animal is treated until total recovery. Accordingly, “treatment” includes amelioration of the symptoms or severity of a particular condition or preventing or otherwise reducing the risk of developing a particular condition.

The term “radiotherapy” or “radiation therapy” is broadly taken to include methods of treatment or therapy with particles and/or energy waves which affect cancerous cells, tumors or related mechanisms and biological processes. In particular the radiation is a high energy wave or particle such as an X-ray, electron, gamma ray or proton used in radiotherapy. Most preferably the wave or particle is an X-ray.

The term “chemotherapeutic agent” is taken broadly to include all drugs, chemicals, compounds, compositions, agents, drugs, polymers, peptides, proteins and the like which affect cancerous cells, tumors or related mechanisms and biological processes.

Preferred isoflavanoid compounds of formula (1) are selected from general formulae (III)-(IX), and more preferably are selected from general formulae (IV)-(IX): 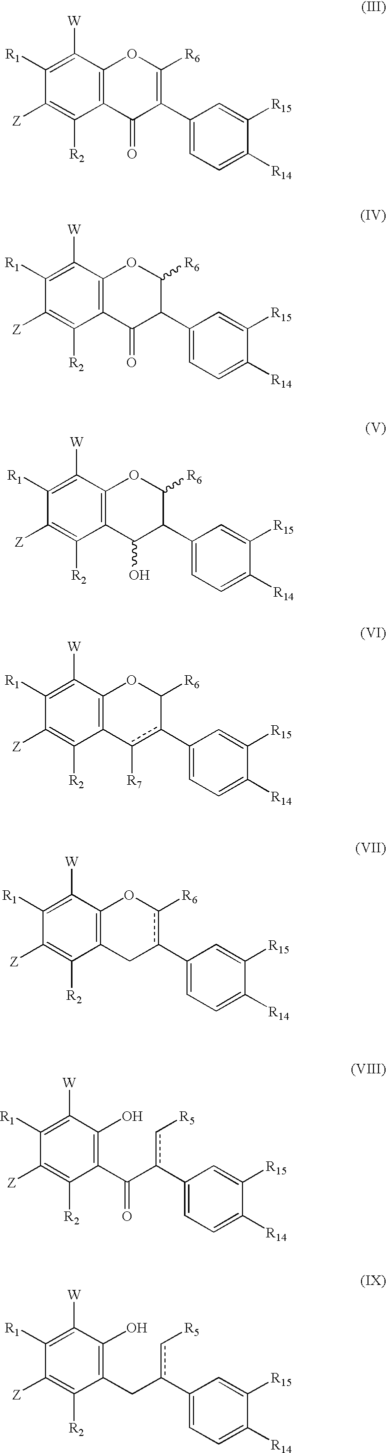
in which
R₁, R₂, R₅, R₆, R₁₄, R₁₅, W and Z are as defined above,
   more preferably
R₁, R₂, R₁₄, R₁₅, W and Z are independently hydrogen, hydroxy, OR₉, OC(O)R₁₀, C(O)R₁₀, COOH, CO₂R₁₀, alkyl, haloalkyl, arylalkyl, aryl, thio, alkylthio, amino, alkylamino, dialkylamino, nitro or halo,
R₅ is hydrogen, C(O)R₁₁ where R₁₁ is hydrogen, alkyl, aryl, or an amino acid, or CO₂R₁₂ where R₁₂ is hydrogen, alkyl or aryl,
R₆ is hydrogen, hydroxy, alkyl, aryl, COR₁₁ where R₁₁ is as previously defined, or CO₂R₁₂ where R₁₂ is as previously defined,
R₇ is hydrogen, C(O)R₁₁ where R₁₁ is as previously defined, alkyl, haloalkyl, alkenyl, aryl, haloaryl, alkylaryl, alkoxyaryl, arylalkyl or Si(R₁₃)₃ where each R₁₃ is independently hydrogen, alkyl or aryl,
R₉ is alkyl, haloalkyl, arylalkyl, or C(O)R₁₁ where R₁₁ is as previously defined, and
R₁₀ is hydrogen, alkyl, amino, aryl, an amino acid, alkylamino or dialkylamino,
   more preferably
R₁ and R₁₄ are independently hydroxy, OR₉, OC(O)R₁₀ or halo,
R₂, R₁₅, W and Z are independently hydrogen, hydroxy, OR₉, OC(O)R₁₀, C(O)R₁₀, COOH, CO₂R₁₀, alkyl, haloalkyl, or halo,
R₅ is hydrogen, C(O)R₁₁ where R₁₁ is hydrogen or alkyl, or CO₂R₁₂ where R₁₂ is hydrogen or alkyl,
R₆ is hydrogen or hydroxy,
R₇ is hydrogen, C(O)R₁₁ where R₁₁ is hydrogen or alkyl, alkyl, haloalkyl, alkenyl, aryl, haloaryl, alkylaryl, alkoxyaryl, arylalkyl or Si(R₁₃)₃ where each R₁₃ is independently hydrogen, alkyl or aryl,
R₉ is alkyl, arylalkyl or C(O)R₁₁ where R₁₁ is as previously defined, and
R₁₀ is hydrogen or alkyl,
   and more preferably
R₁ and R₁₄ are independently hydroxy, methoxy, benzyloxy, acetyloxy or chloro,
R₂, R₁₅, W and Z are independently hydrogen, hydroxy, methoxy, benzyloxy, acetyloxy, methyl, trifluoromethyl or chloro,
R₅ is hydrogen or CO₂R₁₂ where R₁₂ is hydrogen or methyl,
R₆ is hydrogen, and
R₇ is hydrogen, methyl, ethyl, trifluoromethyl, phenyl, halophenyl, methylphenyl, ethylphenyl, methoxyphenyl or phenylmethyl.

Particularly preferred isoflavonoid compounds of formula (I) and pharmaceutically acceptable salts thereof are selected from:

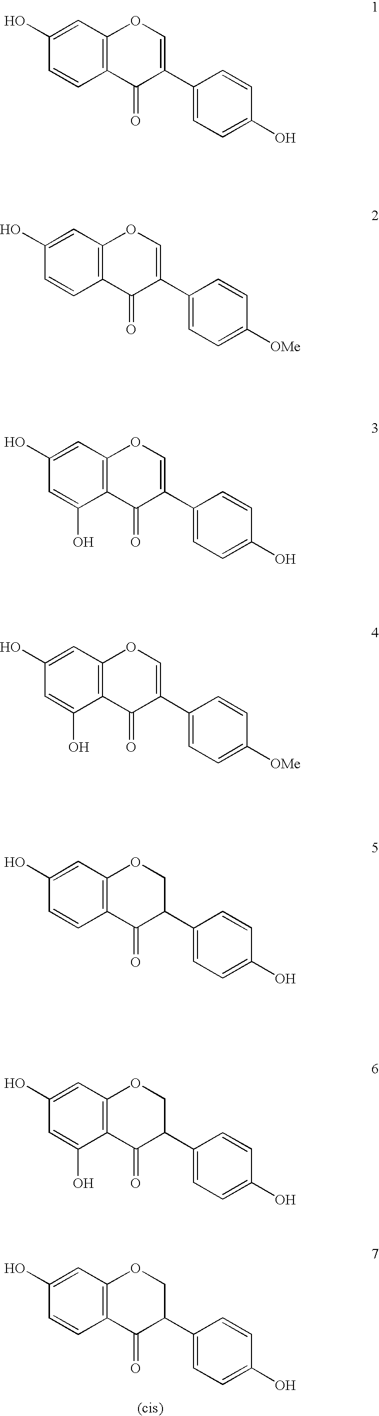

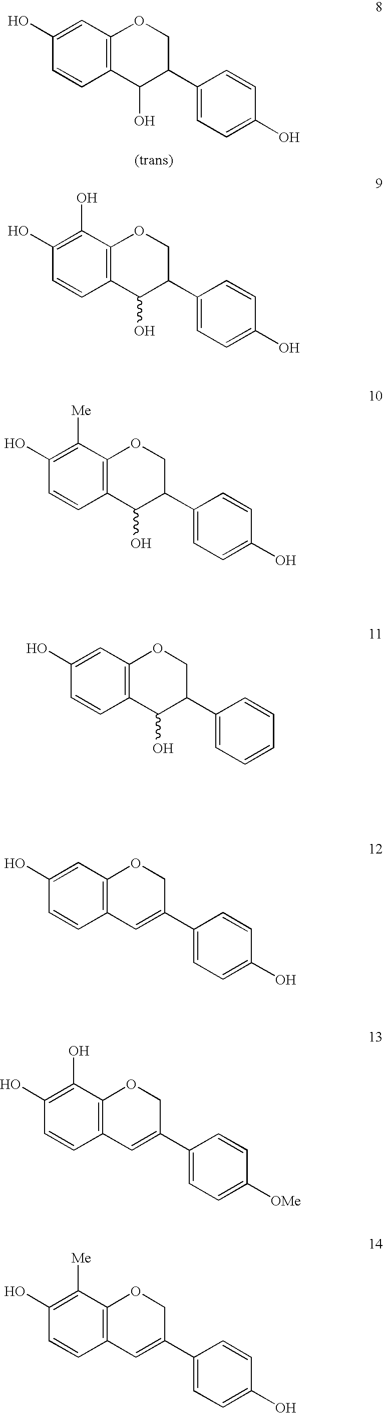

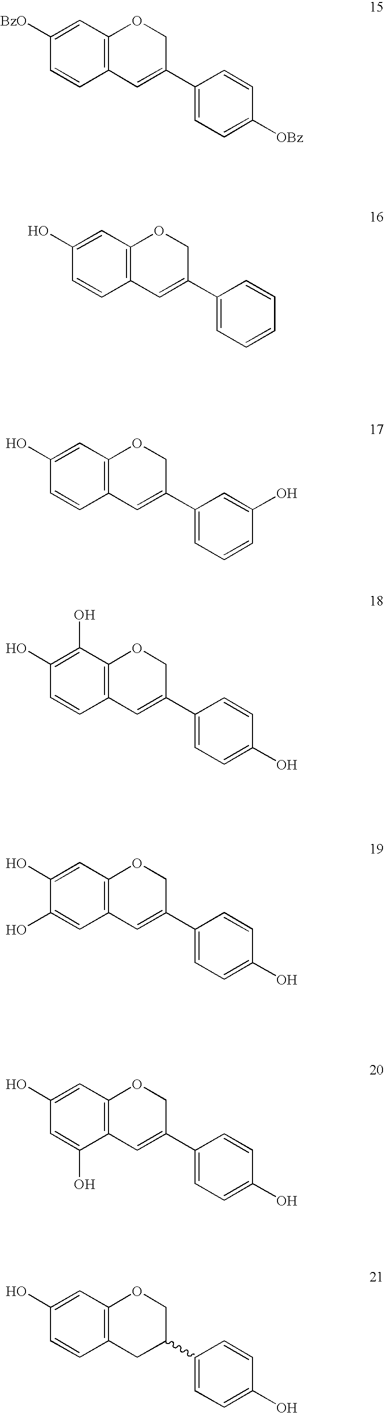

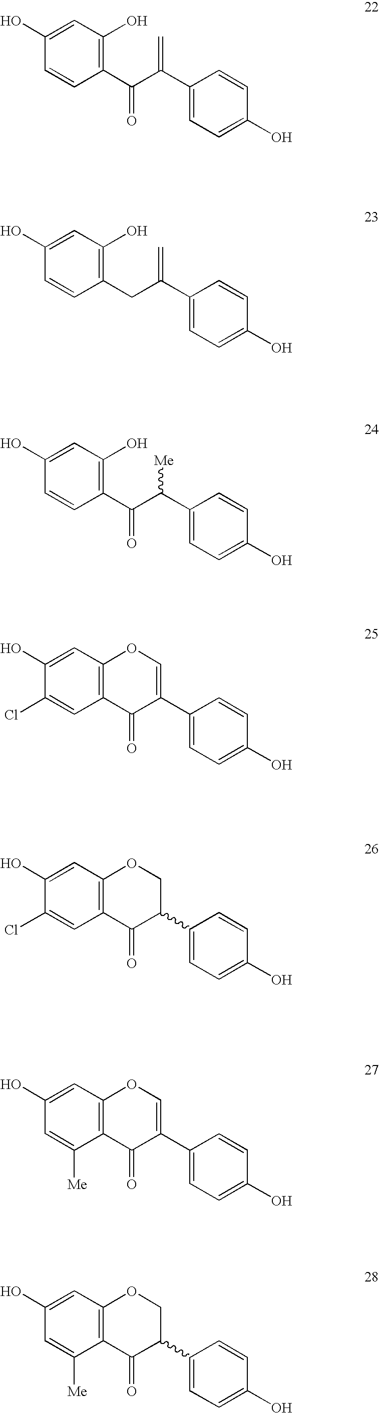

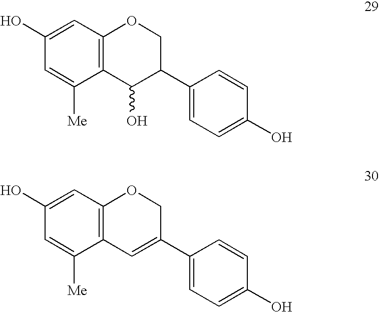

In a further embodiment the preferred isoflavonoid compounds are the isoflav-3-ene and isoflavan compounds of general formula (VI), and specific mention can be made of compounds 12-21 and 30 above in addition to the following compounds and pharmaceutically acceptable salts thereof:

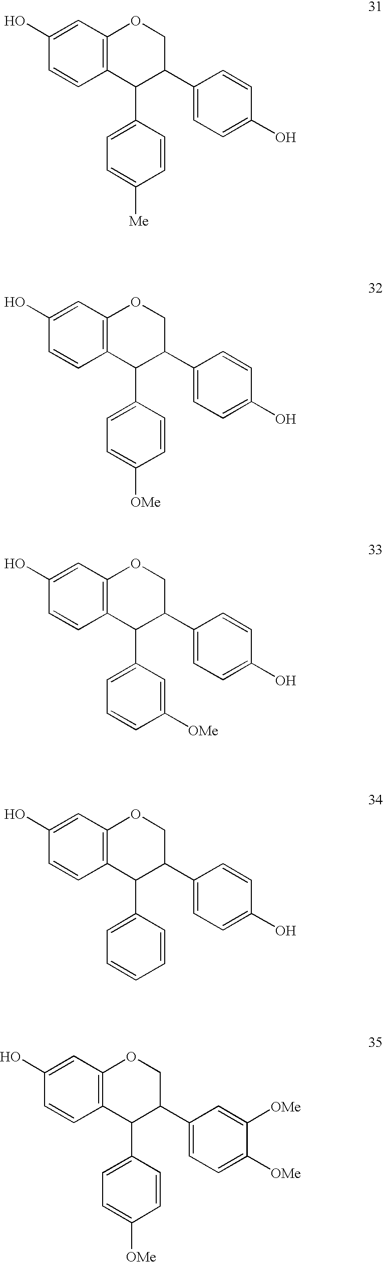

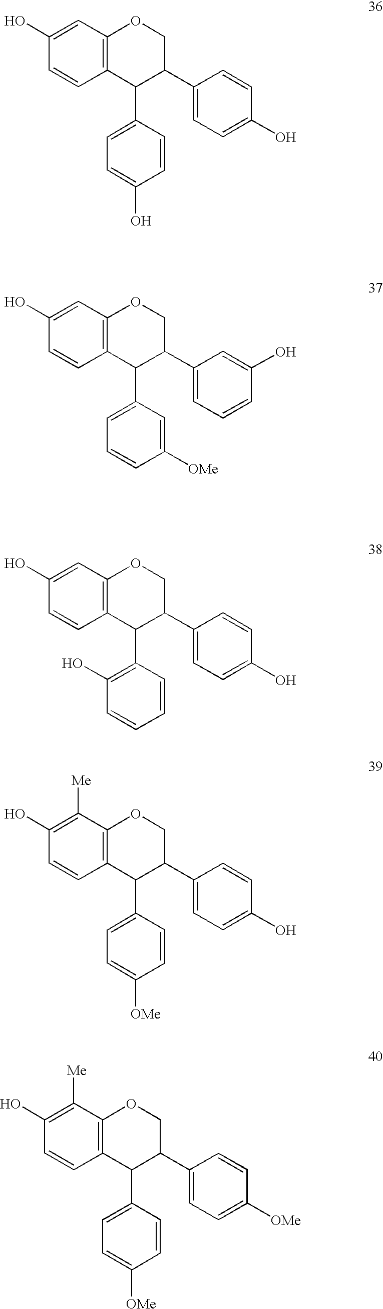

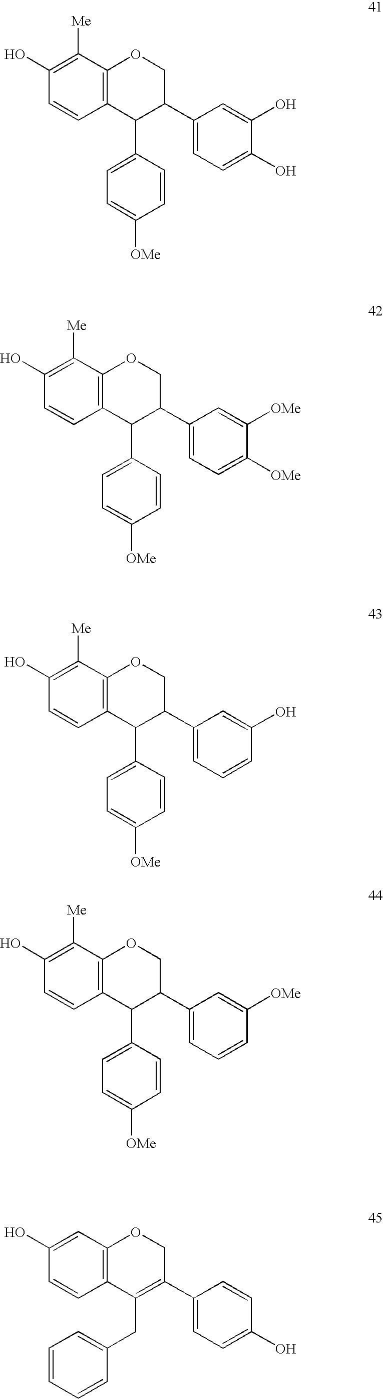

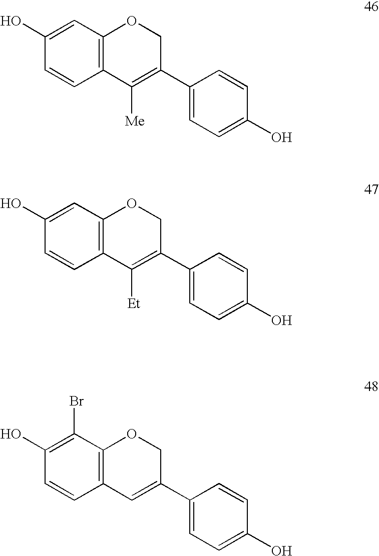

In a most preferred embodiment of the invention the isoflavonoid compound is selected from compounds 12 (dehydroequol), 32, 39, 45 and 48. As such, particular reference is made to dehydroequol in the description, Examples which follow and accompanying drawings however this is not to be taken as being unnecessarily limiting on the disclosure of the invention provided herein.

The chemical family of isoflavonoids of which isoflavones such as genistein and isoflavenes such as dehydroequol are examples, represent a promising new are of chemotherapeutics in the prevention and treatment of cancer. The precise basis of the pharmacological action of these compounds is not fully understood, but the outcome is that of cytostasis and cytotoxicity. Of considerable interest with this family of compounds is that fact that they display broad activity against human and animal cancers, and that they are highly selective for cancer cells.

We have shown that chemical members of this isoflavonoid family, such as dehydroequol, are potent anti-cancer agents surprisingly synergise the effect of chemotherapeutics such as cisplatin and gemcitabine, even though these two standard chemotoxic drugs have quite distinct anti-cancer effects within the cell.

More surprisingly the present inventors have found that the isoflavones or derivatives increase or restore the sensitivity of cancer cells and tumors to the effects of radiation therapy.

Treatment regimes may include a single treatment or a course of treatments, called fractions, over several weeks. The fractional treatment is typically given once a day from Monday to Friday, for example, with intermittent rests such as at weekends to help normal cells recover. The actual treatment regime will largely depend on the type of cancer to be treated and the type of radiotherapy to be employed. Those skilled in the art can best determine the most suitable regime for each individual with consideration being given to various factors including the patent's health, progression of disease and type of cancer.

In a preferred embodiment the isoflavones or derivatives thereof are administered prior to radiotherapy. The effect of the pretreatment is to sensitise the cancerous cells or tumors to the effects of the radiation. The isoflavonoid pre-treatment should begin well prior to and/or during the radiotherapy in order to affect the ability of the target cells to resits the radiation. In a broadest preferred embodiment, the pretreatment is for a time and duration sufficient to contact the cancerous cells or tumor with the administered isoflavonoid. This time may typically take 7 days, or 14 days or up to 30 days. In another preferred embodiment the isoflavonoid treatment is 6 days prior to the radiotherapy, or 5 days, or 3 days or 2 days or 1 day prior.

In other circumstances it can be beneficial to administer the isoflavonoids on the day of the radiotherapy which can still have the effect of contributing to cell death by either removing the blockers of apoptosis or to increase the rate of degradation.

Where the treatment includes fractional treatment, the administration of the isoflavonoid may occur at the stated times prior to the first treatment or only some of or each treatment of radiation.

In another preferred embodiment it is found that administration of the isoflavonoids can restore or at least address sensitivity problems which can occur after radiation treatment. In this respect in another preferred embodiment the administration of the isoflavonoids occurs post radiation treatment.

Chemotherapeutic agents are generally grouped as DNA-interactive agents, antimetabolites, tubulin-interactive agents, hormonal agents, other agents such as asparaginase or hydroxyurea. Each of the groups of chemotherapeutic agents can be further divided by type of activity or compound. Chemotherapeutic agents used in combination with the isoflavonoid compound of formula (I) of the present invention, or salts thereof of the present invention, may be selected from any of these groups but are not limited thereto. For a detailed discussion of the chemotherapeutic agents and their method of administration, see Dorr, et al, Cancer Chemotherapy Handbook, 2d edition, pages 15-34, Appleton and Lang (Connecticut, 1994) herein incorporated by reference.

DNA-interactive agents include alkylating agents, e.g. cisplatin, cyclophosphamide, altretamine; DNA strand-breakage agents, such as bleomycin; intercalating topoisomerase II inhibitors, e.g., dactinomycin and doxorubicin); non-intercalating topoisomerase II inhibitors such as, etoposide and teniposide; and the DNA minor groove binder plicamydin, for example.

The alkylating agents form covalent chemical adducts with cellular DNA, RNA, or protein molecules, or with smaller amino acids, glutathione, or similar chemicals. Generally, alkylating agents react with a nucleophilic atom in a cellular constituent, such as an amino, carboxyl, phosphate, or sulfhydryl group in nucleic acids, proteins, amino acids, or in glutathione. The mechanism and the role of these alkylating agents in cancer therapy is not well understood.

Typical alkylating agents include, but are not limited to, nitrogen mustards, such as chlorambucil, cyclophosphamide, isofamide, mechlorethamine, melphalan, uracil mustard; aziridine such as thiotepa; methanesulphonate esters such as busulfan; nitroso ureas, such as carmustine, lomustine, streptozocin; platinum complexes, such as cisplatin, carboplatin; bioreductive alkylator, such as mitomycin, and procarbazine, dacarbazine and altretamine.

DNA strand breaking agents include bleomycin, for example.

DNA topoisomerase II inhibitors include the following intercalators, such as amsacrine, dactinomycin, daunorubicin, doxorubicin (adriamycin), idarubicin, and mitoxantrone; nonintercalators, such as etoposide and teniposide, for example.

A DNA minor groove binder is plicamycin, for example.

Antimetabolites interfere with the production of nucleic acids by one of two major mechanisms. Certain drugs inhibit production of deoxyribonucleoside triphosphates that are the immediate precursors for DNA synthesis, thus inhibiting DNA replication. Certain of the compounds are analogues of purines or pyrimidines and are incorporated in anabolic nucleotide pathways. These analogues are then substituted into DNA or RNA instead of their normal counterparts.

Antimetabolites useful herein include, but are not limited to, folate antagonists such as methotrexate and trimetrexate; pyrimidine antagonists, such as fluorouracil, fluorodeoxyuridine, CB3717, azacitidine, cytarabine, and floxuridine; purine antagonists include mercaptopurine, 6-thioguanine, fludarabine, pentostatin; and ribonucleotide reductase inhibitors include hydroxyurea.

Tubulin interactive agents act by binding to specific sites on tubulin, a protein that polymerizes to form cellular microtubules. Microtubules are critical cell structure units. When the interactive agents bind the protein, the cell can not form microtubules. Tubulin interactive agents include vincristine and vinblastine, both alkaloids and paclitaxel (Taxol), for example.

Hormonal agents are also useful in the treatment of cancers and tumors. They are used in hormonally susceptible tumors and are usually derived from natural sources. Hormonal agents include, but are not limited to, estrogens, conjugated estrogens and ethinyl estradiol and diethylstilbesterol, chlortrianisen and idenestrol; progestins such as hydroxyprogesterone caproate, medroxyprogesterone, and megestrol; and androgens such as testosterone, testosterone propionate; fluoxymesterone, and methyltestosterone.

Adrenal corticosteroids are derived from natural adrenal cortisol or hydrocortisone. They are used because of their anti-inflammatory benefits as well as the ability of some to inhibit mitotic divisions and to halt DNA synthesis. These compounds include, but are not limited to, prednisone, dexamethasone, methylprednisolone, and prednisolone.

Leutinizing hormone releasing hormone agents or gonadotropin-releasing hormone antagonists are used primarily the treatment of prostate cancer. These include leuprolide acetate and goserelin acetate. They prevent the biosynthesis of steroids in the testes.

Antihormonal antigens include, for example, antiestrogenic agents such as tamoxifen, antiandrogen agents such as flutamide; and antiadrenal agents such as mitotane and aminoglutethimide.

Further agents include the following: hydroxyurea appears to act primarily through inhibition of the enzyme ribonucleotide reductase, and asparaginase is an enzyme which converts asparagine to nonfunctional aspartic acid and thus blocks protein synthesis in the tumor.

Preferred chemotherapeutic agents are cisplatin, carboplatin, taxol (paclitaxel), fluorouracil, fluxuridine, cyclophosphamide ifosfamide, hexamethylmelamine, estramustine, mitomycin, and docetaxel.

Compounds of formula (I) also exhibit chemotherapeutic activity and in this regard particular reference can be made to dehydroequol, Cpd. 12, and to Cpds. 32 and 39.

Preferred bidentate and tridentate platinum ligands of the present invention include those commonly known in the art. For example, suitable bidentate ligands may be selected from ethylene-1,2-diamine and 1,10-phenathraline and other ligands well known in the art.

Compounds of the present invention have particular application in the treatment of diseases associated with or resulting from estrogenic effects, androgenic effects, vasolidatory and spasmodic effects, inflammatory effects and oxidative effects. These effects are further described in International patent application Nos. WO 98/08503 and WO 03/086386.

The amount of compounds of formula (I) which are required in a therapeutic treatment according to the invention will depend upon a number of factors, which include the specific application, the nature of the particular compound used, the condition being treated, the mode of administration and the condition of the patient. Compounds of formula I may be administered in a manner and amount as is conventionally practised. See, for example, Goodman and Gilman, The Pharmacological Basis of Therapeutics, 1299 (7th Edition, 1985). The specific dosage utilised will depend upon the condition being treated, the state of the subject, the route of administration and other well known factors as indicated above. In general, a daily dose per patient may be in the range of 0.1 mg to 10 g; typically from 0.5 mg to 1 g; preferably from 50 mg to 200 mg. Importantly the synergistic relationship of the isoflavanoid compounds of general formula (I) and an anticancer agent allow for significant reductions in dosage regimes of relatively toxic drugs such as cisplatin, paclitaxel and carboplatin for example.

The production of a pharmaceutical composition for the treatment of the therapeutic indications herein described (for convenience hereafter referred to as the “active compounds”) are typically admixed with one or more pharmaceutically or veterinarially acceptable carriers and/or excipients as are well known in the art.

The carrier must, of course, be acceptable in the sense of being compatible with any other ingredients in the formulation and must not be deleterious to the subject. The carrier or excipient may be a solid or a liquid, or both, and is preferably formulated with the compound as a unit-dose, for example, a tablet, which may contain from 0.5% to 59% by weight of the active compound, or up to 100% by weight of the active compound. One or more active compounds may be incorporated in the formulations of the invention, which may be prepared by any of the well known techniques of pharmacy consisting essentially of admixing the components, optionally including one or more accessory ingredients.

The formulations of the invention include those suitable for oral, rectal, optical, buccal (for example, sublingual), parenteral (for example, subcutaneous, intramuscular, intradermal, or intravenous) and transdermal administration, although the most suitable route in any given case will depend on the nature and severity of the condition being treated and on the nature of the particular active compound which is being used.

Formulation suitable for oral administration may be presented in discrete units, such as capsules, sachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture such as to form a unit dosage. For example, a tablet may be prepared by compressing or moulding a powder or granules containing the active compound, optionally with one or more accessory ingredients. Compressed tablets may be prepared by compressing, in a suitable machine, the compound of the free-flowing, such as a powder or granules optionally mixed with a binder, lubricant, inert diluent, and/or surface active/dispersing agent(s). Moulded tablets may be made by moulding, in a suitable machine, the powdered compound moistened with an inert liquid binder.

Formulations suitable for buccal (sublingual) administration include lozenges comprising the active compound in a flavoured base, usually sucrose and acacia or tragacanth; and pastilles comprising the compound in an inert base such as gelatin and glycerin or sucrose and acacia.

Compositions of the present invention suitable for parenteral administration conveniently comprise sterile aqueous preparations of the active compounds, which preparations are preferably isotonic with the blood of the intended recipient. These preparations are preferably administered intravenously, although administration may also be effected by means of subcutaneous, intramuscular, or intradermal injection. Such preparations may conveniently be prepared by admixing the compound with water or a glycine buffer and rendering the resulting solution sterile and isotonic with the blood. Injectable formulations according to the invention generally contain from 0.1% to 60% w/v of active compound(s) and are administered at a rate of 0.1 ml/minute/kg or as appropriate. Parenteral administration is a preferred route of administration for the compounds of the present invention.

Formulations suitable for rectal administration are preferably presented as unit dose suppositories. These may be prepared by admixing the active compound with one or more conventional solid carriers, for example, cocoa butter, and then shaping the resulting mixture.

Formulations or compositions suitable for topical administration to the skin preferably take the form of an ointment, cream, lotion, paste, gel, spray, aerosol, or oil. Carriers which may be used include Vaseline, lanoline, polyethylene glycols, alcohols, and combination of two or more thereof. The active compound is generally present at a concentration of from 0.1% to 0.5% w/w, for example, from 0.5% to 2% w/w. Examples of such compositions include cosmetic skin creams.

Formulations suitable for transdermal administration may be presented as discrete patches adapted to remain in intimate contact with the epidermis of the recipient for a prolonged period of time. Such patches suitably contain the active compound as an optionally buffered aqueous solution of, for example, 0.1 M to 0.2 M concentration with respect to the said active compound.

Formulations suitable for transdermal administration may also be delivered by iontophoresis (see, for example, Pharmaceutical Research 3 (6), 318 (1986)) and typically take the form of an optionally buffered aqueous solution of the active compound. Suitable formulations comprise citrate or bis/tris buffer (pH 6) or ethanol/water and contain from 0.1 M to 0.2 M active ingredient.

The active compounds may be provided in the form of food stuffs, such as being added to, admixed into, coated, combined or otherwise added to a food stuff. The term food stuff is used in its widest possible sense and includes liquid formulations such as drinks including dairy products and other foods, such as health bars, desserts, etc. Food formulations containing compounds of the invention can be readily prepared according to standard practices.

Therapeutic methods, uses and compositions may be for administration to humans or animals, including mammals such as companion and domestic animals (such as dogs and cats) and livestock animals (such as cattle, sheep, pigs and goats), birds (such as chickens, turkeys, ducks) and the like.

The active compound or pharmaceutically acceptable derivatives prodrugs or salts thereof can also be co-administered with other active materials that do not impair the desired action, or with materials that supplement the desired action, such as antibiotics, antifungals, antiinflammatories, or antiviral compounds. The active agent can comprise two or more isoflavones or derivatives thereon in combination or synergistic mixture. The active compounds can also be administered with lipid lowering agents such as probucol and nicotinic acid; platelet aggregation inhibitors such as aspirin; antithrombotic agents such as coumadin; calcium channel blockers such as verapamil, diltiazem, and nifedipine; angiotensin converting enzyme (ACE) inhibitors such as captopril and enalapril, and β-blockers such as propanolol, terbutalol, and labetalol. The compounds can also be administered in combination with nonsteriodal antiinflammatories such as ibuprofen, indomethacin, aspirin, fenoprofen, mefenarnic acid, flufenamic acid and sulindac. The compounds can also be administered with corticosteroids.

The co-administration may be simultaneous or sequential. Simultaneous administration may be effected by the compounds being in the same unit dose, or in individual and discrete unit doses administered at the same or similar time. Sequential administration may be in any order as required and typically will require an ongoing physiological effect of the first or initial active agent to be current when the second or later active agent is administered, especially where a cumulative or synergistic effect is desired.

The isoflavones of formula (I) for use in the present invention may be derived from any number of sources readily identifiable to a person skilled in the art. Preferably, they are obtained by synthetic synthesis. See, for example, Chang et al. (1994) which discloses methods appropriate for the synthesis of various isoflavones. The isoflavones of formula (I) may also be obtained by chemical extraction from plants in which the desired compound either exists naturally or can be obtained by a process of extraction and semi-synthesis.

International Patent Applications WO 98/08503 and WO 00/49009 (which are incorporated herein in their entirety by reference) and references cited therein also provide general synthetic methods for the preparation of isoflavanoid compounds for use in the present invention.

The 3,4 diarylcromans are prepared according to known methods, for example such as those disclosed in U.S. Pat. Nos. 3,340,276 and 3,822,287. Synthesis of particular isomers and inter-conversion methods can be found in U.S. Pat. Nos. 3,822,287 and 4,447,622.

The inventors have found a surprising synergy between the compounds of formula (I), and in particular the isoflav-3-ene compounds of formula (VI), with known radiation therapy methods of treatment. The inventors have also found a surprising synergy between the compounds of formula (I), and in particular the isoflav-3-ene compounds of formula (VI), with chemotherapeutic agents. The isoflavanoid compounds of the invention are found to restore or at least improve chemo-sensitivity to previously resistant cancer cell lines. In addition they are able to protect non-cancer or normal cells or tissue masses from the effects of radiotherapy or chemotherapy by differentiation of cellular characteristics.

In particular, dehydroequol (12) and compounds 32 and 39 are found to exhibit synergistic interaction with interferon-gamma, Fas ligand, TRAIL ligand, growth receptor inhibitors (eg. inhibitors of epithelial growth factor receptor, platelet-derived growth factor receptor, fibroblast growth factor receptor, tumor necrosis factor receptor, insulin-like growth factor receptor). These compounds are thought to be active by having an inhibiting effect on XIAP so as to radiosensitise (or chemosensitise) the cancer cells by upregulating apoptosis and downregulating cell survival

These results are further elucidated in the examples that follow. These results show that combination radiotherapy and/or chemotherapy with the isoflavonoid compounds is useful in the treatment of proliferation of cancer cells and neoplastic tumours by reducing the IC₅₀ of standard chemotherapy. Administration of the isoflavanoid compounds described herein administered either simultaneously, sequentially or as a pre-treatment to standard therapy regimes increases the sensitivity of cancer cells and tumours to chemotoxic agents and to radiotherapy.

The invention is further described with reference to the following non-limiting examples.

### EXAMPLE 1

The effect of a selection of isoflavonoid compounds on various cancer cell lines subjected to radiotherapy was assessed on culture plates. Cell viability was determined using CellTiter®. Apoptosis was evaluated using Hoechst 33342 dye.

Results show that cancer cell lines to human breast, prostate, ovarian, pancreatic and cervical cancers are radiosensitised by both pre-treatment (1 day and 8 hour) and sequential/post-treatment (−30 min/+4 h) regimes with dehydroequol (Cpd. 12) and Cpds. 32 and 39 (at 2 and 10 μM).

Likewise, some other isoflavonoids, Cpds. 7 and 8, exhibit similar results but to differing degrees across the range of human cancer cell lines.

It was surprisingly found that the cellular treatment with isoflavones act as a radiosensitiser to the cancer cell lines.

The results suggest that in a main mechanistic pathway the isoflavonoid compounds inhibit XIAP, thereby greatly decreasing cell survival following irradiation providing better tumor control in the models studied. In addition the action of these isoflavonoids on standard radioresistant cells is found to make them more susceptible to radiotherapy than the control group.

These examples highlight the utility of the isoflavonoid compounds of formula (I) in combination with radiotherapy as therapeutic agents for increasing sensitivity or restoring sensitivity to resistant cancer cells and tumours and to inducing sensitivity to radioresistant cancer cells.

The compounds of formula I are also useful for the general down regulation of cell proliferation and the treatment, amelioration, defence against, prophylaxis and/or prevention of the therapeutic indications.

Those skilled in the art will appreciate that the invention described herein is susceptible to variations and modifications other than those specifically described. It is to be understood that the invention includes all such variations and modifications.

The inventions also includes all of the steps, features, compositions and compounds referred to or indicated in the specification, individually or collectively, and any and all combinations of any two or more of said steps or features.

The reference to any prior art in this specification is not, and should not be taken as, an acknowledgment or any form of suggestion that that prior art forms part of the common general knowledge in the field of endeavour.

1-12. (canceled)

13. A method of increasing or restoring the sensitivity of cancer cells or a tumour to radiotherapy by contacting said cells or tumour with an isoflavonoid compound of formula (I) or a pharmaceutically acceptable salt thereof as herein defined.

14. A method of increasing or restoring the sensitivity of cancer cells or a tumour to chemotherapy by contacting said cells or tumour with an isoflavonoid compound of formula (I) or a pharmaceutically acceptable salt thereof.

15. A method of protecting normal cells or a tissue mass from the effects of radiotherapy and/or chemotherapy by contacting said cells or tissue mass with an isoflavonoid compound of formula (I) or a pharmaceutically acceptable salt thereof.

16. A method of therapeutic treatment of a subject undergoing radiotherapy, or about to undergo radiotherapy, comprising administering to said subject a therapeutically effective amount of a compound of formula (I) or a pharmaceutically acceptable salt thereof.

17. The method of claim 16, wherein the administration occurs prior to radiotherapy.

18. The method of claim 17, wherein the administration occurs after radioresistance has developed in the subject from prior radiation treatments.

19. A method of therapeutic treatment of a subject with cancer comprising administering to said subject a therapeutically effective amount of a compound of formula (I) or a pharmaceutically acceptable salt thereof and a chemotherapeutic agent.

20. The method of claim 19, wherein the administration occurs prior to chemotherapy.

21. The method of claim 20, wherein the administration occurs after chemoresistance has developed in the subject from prior chemotherapy treatments.

22. The method according to claim 16, wherein the condition being treated is cancer that is displaying malignant characteristics, earlier stages of cancer such as pre-malignant lesions (eg. atypia, dysplasia, intra-epitelial neoplasia) and benign cancers.

23. A method according to claim 14, wherein a chemotherapeutic agent is administered to a subject with cancer in combination with isoflavonoid compound of the formula (I).

24. A method according to claim 23, wherein said chemotherapeutic agent is administered simultaneously or sequentially with a compound of the formula (I).

25. A method according to claim 14, wherein the condition being treated is cancer that is displaying malignant characteristics.

26. A method according to claim 14, wherein the condition being treated is early stage cancer, such as pre-malignant lesions.

27. A method according to claim 14, wherein the condition being treated is benign cancer.

28. A method according to claim 14, wherein said isoflavonoid compound of the formula (I) is administered after resistance, either inherent or acquired, to the chemotherapeutic agent is observed in a patient with cancer.

29. A method according to claim 14, wherein said cancer cells or tumour are selected from human breast, prostate, ovarian, pancreatic and cervical cancers.

30. A method according to claim 14, wherein said compounds of the formula (I) are selected from compounds 1 through 48 as herein described.

31. A method according to claim 30, wherein said compounds of the formula (I) are selected from compounds 12 (dehydroequol), 32, 39, 45 and 48 and pharmaceutically acceptable salts thereof.

32. A method according to claim 31, wherein said chemotherapeutic agent is selected from the group of DNA-interactive agents, antimetabolites, tubulin-interactive agents and hormonal agents.

33. A method according to claim 32, wherein said chemotherapeutic agents are selected from cisplatin, carboplatin, taxol, fluorouracil, fluxuridine, cyclophosphamide ifosfamide, hexamethylmelamine, estramustine, mitomycin and docetaxel.

34. A chemotherapeutic composition for the enhancement of sensitivity of cancer cells which comprises an isoflavonoid compound of the formula (I) or a pharmaceutically acceptable salt thereof and a chemotherapeutic agent.

35. The composition according to claim 34, wherein said chemotherapeutic agent is selected from DNA-interactive agents, antimetabolites, tubulin-interactive agents and hormonal agents.

36. The composition according to claim 34, wherein said chemotherapeutic compound is selected from cisplatin, carboplatin, taxol, fluorouracil, fluxuridine, cyclophosphamide ifosfamide, hexamethylmelamine, estramustine, mitomycin and docetaxel.

37. The composition according to claim 34, wherein the said isoflavonoid compound of the formula (I) comprises one or more of compound 1 through 48 as herein described.

38. The composition according to claim 34, wherein said compounds of the formula (I) are selected from compounds 12 (dehydroequol), 32, 39, 45 and 48.
